(19) United States
(12) Patent Application Publication
(10) Pub. No.: US 2021/0282779 A1
(43) Pub. Date: Sep. 16, 2021

(54) ADAPTER ASSEMBLY FOR SURGICAL STAPLING INSTRUMENTS HAVING A CONTACT SWITCH

(71) Applicant: Covidien LP, Mansfield, MA (US)

(72) Inventor: Joseph Eisinger, Northford, CT (US)

(21) Appl. No.: 17/192,289

(22) Filed: Mar. 4, 2021

Related U.S. Application Data
(60) Provisional application No. 62/989,237, filed on Mar. 13, 2020.

(51) Int. Cl. A61B 17/115

(52) U.S. Cl. CPC A61B 17/1155; A61B 2017/00831

(57) ABSTRACT

A surgical stapling instrument includes an anvil assembly, a shell assembly, and an adapter assembly. The anvil assembly includes an anvil head and an anvil center rod. The shell assembly includes an annular staple cartridge including a plurality of staples. The adapter assembly includes a tubular shaft supporting the shell assembly at a distal portion of the tubular shaft, a trocar assembly, a cable assembly, and a switch. The trocar assembly includes a trocar detachably supporting the anvil center rod thereon, a lead screw adapted to be coupled to an actuator for rotational input, and a first member rotatably supporting the lead screw. The cable assembly includes a cable operatively coupled to the actuator and a coupling member secured with the cable for concomitant rotation therewith. The coupling member detachably engages the lead screw. The switch is transitionable to a closed state when the first member engages the switch.

### CROSS-REFERENCE TO RELATED APPLICATION

This application claims the benefit of and priority to U.S. Provisional Patent Application Ser. No. 62/989,237, filed Mar. 13, 2020, the disclosure of which is incorporated by reference herein in its entirety.

### FIELD

The disclosure relates generally to surgical stapling instruments, and more particularly, to an adapter assembly for surgical stapling instruments having a contact switch.

### BACKGROUND

Anastomosis is the surgical joining of separate hollow organ sections. Typically, an anastomosis procedure follows surgery in which a diseased or defective section of hollow tissue is removed, and the end sections are stapled via a surgical stapling instrument. Depending on the desired anastomosis procedure, the end sections may be joined by circular or side-to-side organ reconstruction methods, for instance.

In a circular anastomosis procedure, the two ends of the organ sections are joined by means of a surgical stapling instrument which drives a circular array of staples through the end section of each organ section and simultaneously cores any tissue interior of the driven circular array of staples to free the tubular passage. Typically, these surgical stapling instruments include an elongated body portion having a handle portion at a proximal end to actuate the surgical stapling instrument and a staple holding component disposed at a distal end. An anvil assembly including an anvil retention rod with an attached anvil head is mounted to a trocar assembly at the distal end of the surgical stapling instrument adjacent the staple-holding component. Opposed end portions of tissue of the hollow organ(s) to be stapled are clamped between the anvil head and the staple holding component. The clamped tissue is stapled by driving one or more staples from the staple holding component so that the ends of the staples pass through the tissue and are formed by the anvil head. An annular knife is advanced to core tissue within the hollow organ to free a tubular passage within the organ.

Besides anastomosis of hollow organs, surgical stapling instruments for performing circular anastomosis have been used to treat internal hemorrhoids in the rectum. Typically, during use of a surgical stapling instrument for hemorrhoid treatment, the anvil head and the staple holding-component of the surgical stapling instrument are inserted through the anus and into the rectum with the anvil head and the staple-holding component in an open or spaced part position. Thereafter, a purse string suture is used to pull the internal hemorrhoidal tissue towards the anvil rod. Next, the anvil head and staple-holding component are approximated to clamp the hemorrhoidal tissue between the anvil head and the staple holding component. During the approximation of the anvil head and the staple-holding component, the trocar assembly is engaged with the anvil retention rod. The surgical stapling instrument is fired to remove the hemorrhoidal tissue and staple the tissue.

### SUMMARY

In accordance with the disclosure, a surgical stapling instrument includes an anvil assembly, a shell assembly, and an adapter assembly. The anvil assembly includes an anvil head and an anvil center rod extending proximally from the anvil head. The shell assembly includes an annular staple cartridge including a plurality of staples. The adapter assembly includes a tubular shaft supporting the shell assembly at a distal portion of the tubular shaft, a trocar assembly transitionable between an extended configuration and a retracted configuration, a cable assembly, and a normally open switch. The trocar assembly includes a trocar detachably supporting the anvil center rod thereon, a lead screw adapted to be coupled to an actuator for rotational input, and a first member rotatably supporting the lead screw. The cable assembly includes a cable operatively coupled to the actuator and a coupling member secured with the cable for concomitant rotation therewith. The coupling member detachably engages the lead screw. The normally open switch is transitionable to a closed state when the first member engages the normally open switch.

In an aspect, the first member may include a proximal engaging portion configured to transition the normally open switch to the closed state when in contact with the normally open switch.

In another aspect, a proximal portion of the lead screw may extend through the proximal engaging portion of the first member.

In yet another aspect, the tubular shaft of the adapter assembly may include a support member rotatably supporting the coupling member of the cable assembly.

In an aspect, the support member of the adapter assembly may include the normally open switch.

In another aspect, the proximal portion of the lead screw may extend through the normally open switch when the proximal portion of the lead screw is coupled to the coupling member of the cable assembly.

In yet another aspect, the support member of the adapter assembly may be formed of an electrically insulating material.

In still yet another aspect, the proximal engaging portion of the first member may be formed of a conductive metal.

In still yet another aspect, a portion of the coupling member of the cable assembly may be flush with a proximal surface of the support member of the adapter assembly.

In an aspect, a portion of the normally open switch may be on the proximal surface of the support member of the adapter assembly.

In another aspect, the lead screw may include an annular protrusion configured to be received in a circular groove defined in an inner surface of the first member to inhibit axial displacement of the lead screw during rotation thereof.

In accordance with another aspect of the disclosure, an adapter assembly for use with a surgical stapling instrument includes a tubular shaft supporting a shell assembly of the surgical stapling instrument at a distal portion of the tubular shaft, a trocar assembly attachable to an anvil assembly of the surgical stapling instrument, a cable assembly, and a contact switch. The trocar assembly is transitionable between an extended configuration and a retracted configuration. The trocar assembly includes a trocar detachably supporting the anvil center rod thereon, a lead screw adapted to be coupled to an actuator for rotational input, and a first member rotatably supporting the lead screw. The cable assembly includes a cable operatively coupled to the actuator and a coupling member secured with the cable for concomitant rotation therewith. The coupling member detachably engages the lead screw of the trocar assembly. The contact switch is configured to transition from an open state to a closed state when the first member engages the contact switch.

In an aspect, the coupling member may define a bore configured to receive a proximal portion of the lead screw.

In another aspect, the first member may include a proximal engaging portion configured to engage the contact switch to transition the contact switch to the closed state.

In yet another aspect, a proximal portion of the lead screw may extend through the proximal engaging portion of the first member.

In still yet another aspect, the proximal portion of the lead screw may extend through the contact switch when the proximal portion of the lead screw is coupled to the coupling member of the cable assembly.

In still yet another aspect, the tubular shaft of the adapter assembly may include a support member rotatably supporting the coupling member of the cable assembly and including the contact switch.

In still yet another aspect, the contact switch may include a metal insert exposed to a proximal surface of the support member.

In still yet another aspect, a portion of the coupling member of the cable assembly may be coplanar with the metal insert of the contact switch.

In another aspect, the trocar assembly may further include a second member operatively coupled to the lead screw such that rotation of the lead screw causes axial displacement of the second member relative to the first member. The second member may support the trocar.

### BRIEF DESCRIPTION OF DRAWINGS

An adapter assembly for use with a surgical stapling instrument is disclosed herein with reference to the drawings, wherein:

FIG. 1 is a perspective view of a surgical stapling instrument in accordance with the disclosure;

FIG. 2 is a perspective view of an anvil assembly and a shell assembly of the surgical stapling instrument of FIG. 1;

FIG. 3 is a cross-sectional view of a trocar assembly of an adapter of the surgical stapling instrument of FIG. 1;

FIG. 4 is a partial cross-sectional view of an adapter assembly of the surgical stapling instrument of FIG. 1, illustrating the trocar assembly attached to a coupling member of a cable assembly; and

FIG. 5 is a perspective view of the anvil and the shell assembly of FIG. 2 when the trocar assembly is in a full contact with the coupling member of the adapter assembly.

### DETAILED DESCRIPTION

A surgical stapling instrument is described in detail with reference to the drawings, wherein like reference numerals designate corresponding elements in each of the several views. As used herein, the term “distal” refers to that portion of the instrument, or component thereof which is farther from the user during customary use of the instrument while the term “proximal” refers to that portion of the instrument or component thereof which is closer to the user during customary use of the instrument.

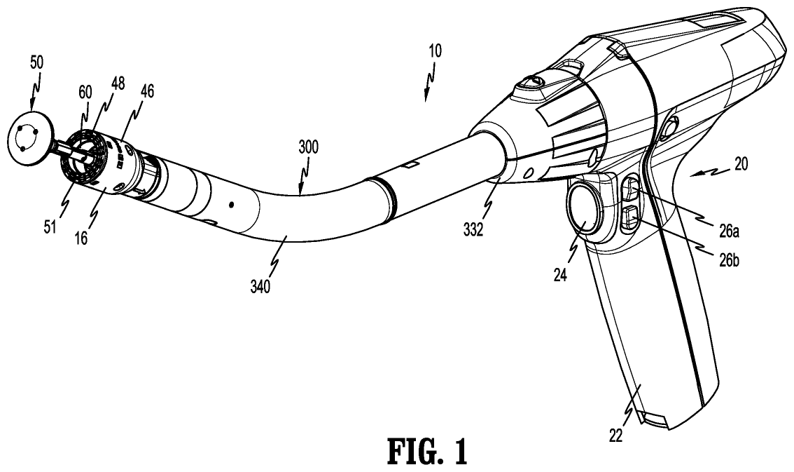

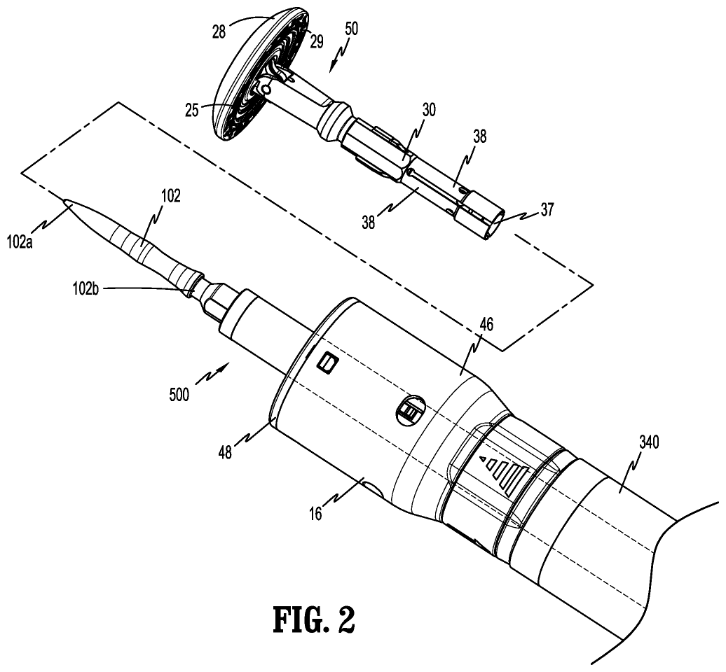

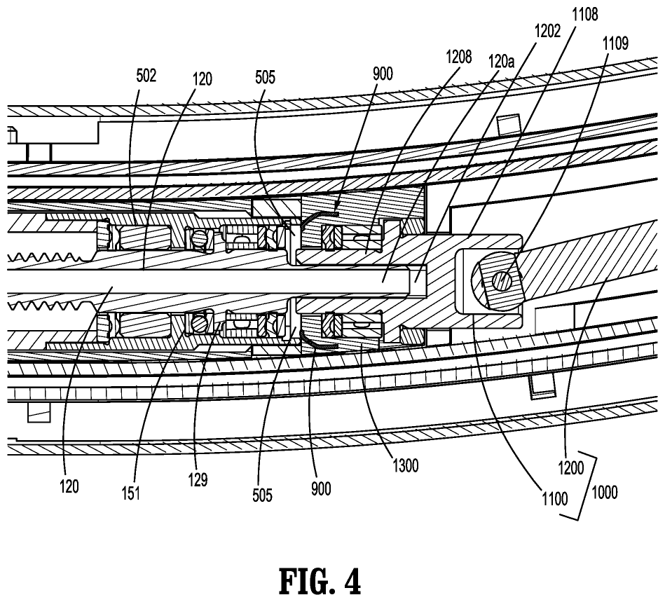

With reference to FIGS. 1 and 2, an adapter assembly for use with a surgical instrument, in the form of a surgical stapling instrument 10 is shown generally as 300. The adapter assembly 300 includes a contact switch 900 (FIG. 4) used to provide a feedback to the user about the status of a connection between a trocar assembly 500 and a coupling member 1100 (FIG. 4) of a cable assembly 1000 providing rotational input to the trocar assembly 500, as will be discussed. The surgical stapling instrument 10 is a circular stapling instrument including a handle assembly 20, the adapter assembly 300 extending distally from the handle assembly 20 and including the trocar assembly 500, a shell assembly 16 supported on a distal portion of the adapter assembly 300, and an anvil assembly 50 operatively coupled to the handle assembly 20.

The handle assembly 20 is illustrated as a powered assembly and includes a stationary grip 22, an actuation button 24 for controlling firing of staples (not shown) from an annular staple cartridge 48 of the shell assembly 16, and approximation buttons 26a, 26b for controlling axial displacement of the anvil assembly 50 towards and away from the shell assembly 16. For a detailed description of the structure and function of exemplary powered handle assemblies, reference may be made to U.S. Patent Application Publication Nos. 2020/0015820 and 2019/0343517, the entire contents of each of which are incorporated herein by reference. Although the present disclosure illustrates a powered assembly, it is envisioned that the advantages of the disclosure as described in detail below are also applicable to surgical stapling instruments having manually operated handle and body assemblies or robotically actuated surgical instruments. U.S. Pat. No. 7,303,106 (the '106 patent) discloses an example of a surgical stapling instrument including a manually actuated handle assembly and is incorporated herein by reference in its entirety. It is also envisioned that the disclosed stapling instrument can be supported on a robotic system and need not include a handle assembly.

With continued reference to FIGS. 1 and 2, the adapter assembly 300 includes an interface portion 332 detachably coupled to the handle assembly 20, a tubular shaft 340 extending distally from the interface portion 332, and the trocar assembly 500 operatively supported within the tubular shaft 340. The shell assembly 16 is supported on a distal portion of the tubular shaft 340 and includes a shell housing 46 and an annular staple cartridge 48 that defines annular rows of staple receiving pockets 51. In particular, the shell assembly 16 may be releasably coupled to the distal portion of the tubular shaft 340 to facilitate replacement of the annular staple cartridge 48 after each use.

Each of the staple receiving pockets 51 supports a staple (not shown) that can be fired from the annular staple cartridge 48 via actuation of the actuation button 24 of the handle assembly 20 and formed within staple forming pockets 25 of a staple forming surface 29 of an anvil head 28 of the anvil assembly 50. The shell housing 46 of the shell assembly 16 defines an annular cavity 60. The annular cavity 60 supports a staple pusher (not shown) and an annular knife (not shown) such that the staple pusher and the annular knife are movable in relation to the annular staple cartridge 48 to eject the staples from the annular staple cartridge 48 and to dissect or cut tissue positioned within an annulus defined by the annular staple cartridge 48. For a detailed description of the structure and function of the exemplary shell assemblies reference may be made to the '106 patent.

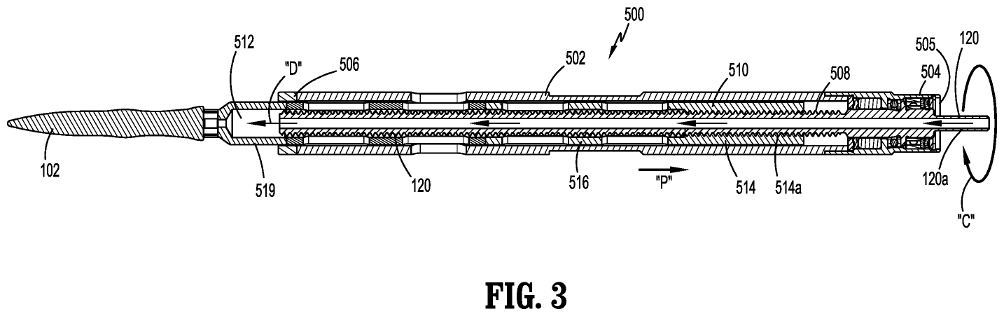

With particular reference to FIG. 2, the anvil assembly 50 includes an anvil head 28 and an anvil center rod 30. The anvil head 28 includes the staple forming surface 29 that includes the staple forming pockets 25. The anvil center rod 30 includes a plurality of resilient fingers 38 defining a longitudinal bore 37 that is dimensioned to receive and releasably engage a trocar 102 of the trocar assembly 500. In an aspect, the anvil head 28 may be pivotally coupled to the anvil center rod 30 and may be movable between an operative position for forming staples and a tilted, reduced profile position. The anvil assembly 50 may be releasably coupled to the trocar assembly 500 for concomitant axial displacement therewith relative to the shell assembly 16 (FIG. 1) by activating an actuator (not shown) such as, e.g., an electric motor, in the handle assembly 20 (FIG. 1). The trocar 102 includes a distal portion 102a that is tapered and a proximal portion 102b has a diameter larger than a diameter of the distal portion 102a. The distal portion 102a is detachably received within the longitudinal bore 37 that is defined by the plurality resilient fingers 38 of the anvil assembly 50. Rotational input to the trocar assembly 500 (FIG. 3) transitions the anvil assembly 50 between a spaced apart configuration and an approximated configuration, in which, the staple forming surface 29 of the anvil assembly 50 is in juxtaposed alignment with the annular staple cartridge 48.

With reference now to FIGS. 3 and 4, the trocar assembly 500 includes an outer member 502, an inner member 510 slidably disposed within the outer member 502, a lead screw 120, and the trocar 102. In particular, the outer member 502, the inner member 510, the lead screw 120, and the trocar 102 may be concentrically arranged. The outer member 502 includes proximal and distal end portions 504, 506 and defines a first longitudinal channel 508 therein. In particular, the lead screw 120 is rotatably supported on, e.g., the proximal end portion 504, of the outer member 502. In particular, a proximal portion 120a of the lead screw 120 extends proximally through an engaging portion 505 of the outer member 502. The lead screw 120 includes an annular protrusion 129 configured to be received in a circular groove defined in an inner surface of the outer member 502 to inhibit axial displacement of the lead screw 120 during rotation of the lead screw 120. The outer member 502 may include a circular protrusion 151 extending radially inward from an inner surface thereof. The circular protrusion 151 is configured to rotatably support the lead screw 120 thereon.

With particular reference to FIG. 3, the distal end portion 506 of the outer member 502 defines an opening dimensioned to receive the inner member 510 therethrough to enable relative axial displacement of the inner member 510 within the first longitudinal channel 508 of the outer member 502. The inner member 510 includes an engaging portion 514 and a receiving portion 516 distal of the engaging portion 514. The engaging portion 514 defines a threaded bore 514a that threadably engages the lead screw 120. The receiving portion 516 is configured to support the trocar 102 at a distal end portion 519 of the receiving portion 516. The inner member 510 and the trocar 102 may be integrally formed as a single construct. Alternatively, the inner member 510 and the trocar 102 may be monolithically formed. However, it is contemplated that the trocar 102 may be detachably supported at the distal end portion 519 of the receiving portion 516. The receiving portion 516 of the inner member 510 defines a second longitudinal channel 512 extending along a length thereof. The second longitudinal channel 512 is dimensioned to receive the lead screw 120 therein.

With particular reference to FIG. 4, the lead screw 120 is operatively coupled to an actuator (not shown) such as, e.g., an electric motor, in the handle assembly 20 (FIG. 1) for rotational input. In particular, the cable assembly 1000 operatively couples the lead screw 120 to the actuator for rotational input. The cable assembly 1000 includes a cable 1200 coupled to the actuator, and a coupling member 1100 detachably engaging the proximal portion 120a of the lead screw 120. The proximal portion 120a of the lead screw 120 protrudes proximally through the engaging portion 505 of the outer member 502 of the trocar assembly 500. The cable 1200 may be formed of a flexible material to enable flexion of the cable in, e.g., radial and/or axial, directions. In this manner, the cable 1200 may accommodate the shape and contour of the adapter assembly 300 (FIG. 1).

With continued reference to FIG. 4, the coupling member 1100 is rotatably supported in the adapter assembly 300. In particular, the coupling member 1100 includes a proximal portion 1108 that is secured with the cable 1200 by, e.g., a pin 1109, and a distal portion 1208 defining a bore 1202 configured to detachably receive the proximal portion 120a of the lead screw 120 therein. The proximal portion 120a of the lead screw 120 may have a non-circular cross-section and the bore 1202 may have a complementary shape to reduce slippage therebetween. In addition, the adapter assembly 300 further includes a support member 1300 rotatably supporting the coupling member 1100. In particular, the support member 1300 includes a contact switch 900 that is normally open in the absence of the trocar assembly 500, i.e., until the engaging portion 505 of the outer member 502 of the trocar assembly 500 engages the contact switch 900 of the support member 1300. For example, the support member 1300 may be formed of an electrically insulating material, and the engaging portion 505 may be formed of a conductive metal. The contact switch 900 may include metal inserts configured to engage the engaging portion 505 formed of the conductive metal, which, in turn, transitions the contact switch 900 to a closed state.

In an aspect, a portion of the coupling member 1100 of the cable assembly 1000 may be flush with a proximal surface of the support member 1300. A portion of the contact switch 900 may be exposed to the proximal surface of the support member 1300. For example, the metal inserts of the contact switch 900 may be exposed to the proximal surface of the support member 1300. A portion of the coupling member 1100 of the cable assembly 1000 may be coplanar with the metal inserts of the contact switch 900. The proximal portion 120a of the lead screw 120 extends through the contact switch 900 when the proximal portion 120a is coupled to the coupling member 1100 of the cable assembly 1000.

The adapter 300 or the handle assembly 100 may include a circuit (not shown) that provides a feedback to the clinician based on the full engagement or contact of the engaging portion 505 of the trocar assembly 500 with the contact switch 900. The contact between the engaging portion 505 and the support member 1300 closes the normally open contact switch 900. For example, the circuit may include, e.g., a light emitting diode (LED), that emits different color lights to indicate open or closed state of the circuit based on the attached state of the trocar assembly 500 with the coupling member 1100. Further, the adapter assembly 300 and/or the handle assembly 100 may utilize a printed circuit board (not shown) including a processor that may utilize the state of the circuit, e.g., the attachment of the trocar assembly 500 to the coupling member 1100 may be a prerequisite for activating the actuator in the handle assembly 20.

Under such a configuration, the closed circuit provides a feedback to the clinician or to the processor indicating the state of the attachment of the trocar assembly 500 with the support member 1300. Based on the feedback, the clinician or the processor may provide rotation of the cable assembly 1200 which imparts concomitant rotation to the lead screw 120. Rotation of the lead screw 120 causes axial displacement of the anvil assembly 50. Under such a configuration, when the lead screw 120 engages the threaded bore 514a of engaging portion 514 of the inner member 510 and rotated in the direction of, e.g., an arrow “C”, by activation of the actuator in the handle assembly 20, the inner member 510 is axially displaced in the direction of an arrow “P”. Axial displacement of the inner member 510 imparts concomitant axial displacement to the trocar 102. Rotation of the lead screw 120 in the direction opposite of the arrow “C” causes axial displacement of the inner member 510 and the trocar 102 in the direction an arrow “D” (FIG. 3).

By providing a contact switch 900 in the adapter assembly 300, the clinician is able to determine the attached state of the trocar assembly 500 relative to the coupling member 1100. For example, under such a configuration, when the proximal portion 120a is not fully received in the bore 1202 of the coupling member 1100, the contact switch 900 remains in the open state, as the engaging portion 505 is disengaged from the contact switch 900. Such a feedback is very valuable when the trocar assembly 500 is loaded onto the adapter 300 after the trocar assembly 500 has been reprocessed or sterilized for reuse.

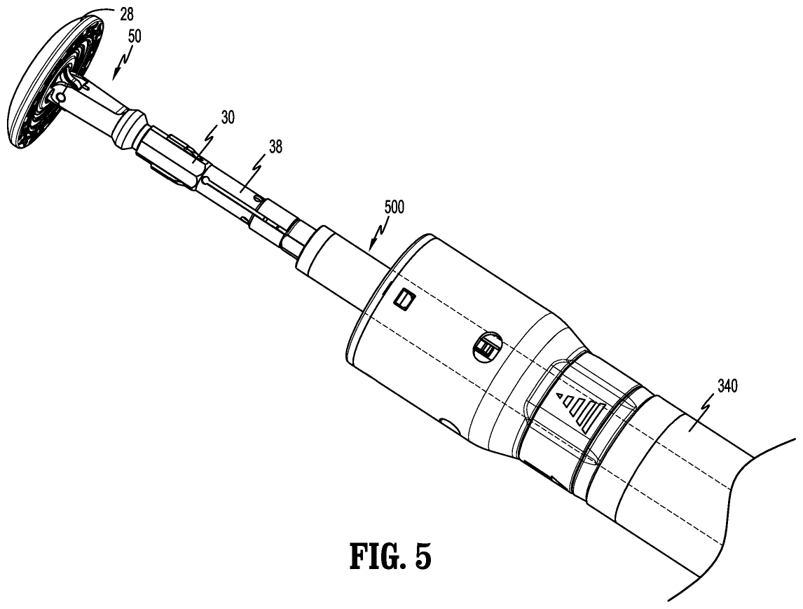

Initially, a first segment of a tubular tissue may be secured to the anvil assembly 50, and a second segment of a tubular tissue may be secured to the trocar assembly 500, by, e.g., purse string suture. The first and second sections of the tubular tissue are placed between the anvil head 28 and the shell assembly 16 to perform anastomosis. At this time, the surgical stapling instrument 10 may be in the spaced apart configuration (FIG. 1). The approximation button 26a may be pressed to transition the anvil head 28 of the anvil assembly 50 to the approximated configuration to clamp tissue between the anvil head 28 and the annular cartridge assembly 48. At this time, the actuator of the handle assembly is activated to provide rotational input to the lead screw 120. Rotation of the lead screw 120 provides axial displacement of the inner member 510 in the direction of the arrows “P” (FIG. 3), which, in turn, retracts the anvil head 28 to the approximated configuration. At this time, tissue is clamped between the anvil head 28 and the shell assembly 16. Thereafter, the actuation button 24 may be pressed to activate an actuator to perform stapling and cutting of tissue disposed between the anvil head 28 and the shell assembly 16. Thereafter, the clinician may press the approximation button 26b to transition the anvil head 28 to the spaced apart configuration. After the surgical procedure, the anvil assembly 50 may be detached from the trocar 102, and the trocar assembly 500 may be detached from the cable assembly 1000 of the adapter assembly 300. Thereafter the anvil assembly 50, the trocar assembly 500, and/or the adapter assembly 300 may be reprocessed or sterilized for reuse. The reprocessed or the sterilized trocar assembly 500 may be loaded onto the adapter assembly 300 (FIG. 5). At this time, the clinician may receive a feedback of the state of contact between the engaging portion 505 of the trocar assembly 500 and the contact switch 900 to determine if the proximal portion 120a of the lead screw 120 of the trocar assembly 500 is fully received in the bore 1202 of the coupling member 1100. For example, less than a full insertion of the proximal portion 120a of the lead screw 120 in the bore 1202 results in, e.g., an open circuit, and alerts the clinician and/or the processor of improper attachment between the trocar assembly 500 and the cable assembly 1000.

Persons skilled in the art will understand that the instruments and methods specifically described herein and illustrated in the accompanying drawings are non-limiting. It is envisioned that the elements and features may be combined with the elements and features of another without departing from the scope of the disclosure. As well, one skilled in the art will appreciate further features and advantages of the disclosure.

What is claimed is:

1. A surgical stapling instrument comprising:
an anvil assembly including an anvil head and an anvil center rod extending proximally from the anvil head;
a shell assembly including an annular staple cartridge including a plurality of staples; and
an adapter assembly including:
a tubular shaft supporting the shell assembly at a distal portion of the tubular shaft;
a trocar assembly transitionable between an extended configuration and a retracted configuration, the trocar assembly including:
a trocar detachably supporting the anvil center rod thereon;
a lead screw adapted to be coupled to an actuator for rotational input; and
a first member rotatably supporting the lead screw;
a cable assembly including:
a cable operatively coupled to the actuator; and
a coupling member secured with the cable for concomitant rotation therewith, the coupling member detachably engaging the lead screw; and
a normally open switch transitionable to a closed state when the first member engages the normally open switch.

2. The surgical stapling instrument according to claim 1, wherein the first member includes a proximal engaging portion configured to transition the normally open switch to the closed state when in contact with the normally open switch.

3. The surgical stapling instrument according to claim 2, wherein a proximal portion of the lead screw extends through the proximal engaging portion of the first member.

4. The surgical stapling instrument according to claim 3, wherein the tubular shaft of the adapter assembly includes a support member rotatably supporting the coupling member of the cable assembly.

5. The surgical stapling instrument according to claim 4, wherein the support member of the adapter assembly includes the normally open switch.

6. The surgical stapling instrument according to claim 5, wherein the proximal portion of the lead screw extends through the normally open switch when the proximal portion of the lead screw is coupled to the coupling member of the cable assembly.

7. The surgical stapling instrument according to claim 4, wherein the support member of the adapter assembly is formed of an electrically insulating material.

8. The surgical stapling instrument according to claim 2, wherein the proximal engaging portion of the first member is formed of a conductive metal.

9. The surgical stapling instrument according to claim 5, wherein a portion of the coupling member of the cable assembly is flush with a proximal surface of the support member of the adapter assembly.

10. The surgical stapling instrument according to claim 9, wherein a portion of the normally open switch is on the proximal surface of the support member of the adapter assembly.

11. The surgical stapling instrument according to claim 1, wherein the lead screw includes an annular protrusion configured to be received in a circular groove defined in an inner surface of the first member to inhibit axial displacement of the lead screw during rotation thereof.

12. An adapter assembly for use with a surgical stapling instrument comprising:
a tubular shaft supporting a shell assembly of the surgical stapling instrument at a distal portion of the tubular shaft;
a trocar assembly attachable to an anvil assembly of the surgical stapling instrument, the trocar assembly transitionable between an extended configuration and a retracted configuration, the trocar assembly including:
a trocar detachably supporting the anvil center rod thereon;
a lead screw adapted to be coupled to an actuator for rotational input; and
a first member rotatably supporting the lead screw;
a cable assembly including:
a cable operatively coupled to the actuator; and
a coupling member secured with the cable for concomitant rotation therewith, the coupling member detachably engaging the lead screw of the trocar assembly; and
a contact switch configured to transition from an open state to a closed state when the first member engages the contact switch.

13. The adapter assembly according to claim 12, wherein the coupling member defines a bore configured to receive a proximal portion of the lead screw.

14. The adapter assembly according to claim 12, wherein the first member includes a proximal engaging portion configured to engage the contact switch to transition the contact switch to the closed state.

15. The adapter assembly according to claim 14, wherein a proximal portion of the lead screw extends through the proximal engaging portion of the first member.

16. The adapter assembly according to claim 15, wherein the proximal portion of the lead screw extends through the contact switch when the proximal portion of the lead screw is coupled to the coupling member of the cable assembly.

17. The adapter assembly according to claim 13, wherein the tubular shaft of the adapter assembly includes a support member rotatably supporting the coupling member of the cable assembly and including the contact switch.

18. The adapter assembly according to claim 17, wherein the contact switch includes a metal insert exposed to a proximal surface of the support member.

19. The adapter assembly according to claim 18, wherein a portion of the coupling member of the cable assembly is coplanar with the metal insert of the contact switch.

20. The adapter assembly according to claim 13, wherein the trocar assembly further includes a second member operatively coupled to the lead screw such that rotation of the lead screw causes axial displacement of the second member relative to the first member, the second member supporting the trocar.
